(19) United States
(12) Patent Application Publication
(10) Pub. No.: US 2025/0177724 A1
(43) Pub. Date: Jun. 5, 2025

(54) PERCUTANEOUS BLOOD PUMP WITH MOTOR CONNECTION

(71) Applicant: Boston Scientific Scimed, Inc., Maple Grove, MN (US)

(72) Inventor: Lloyd Radman, Blaine, MN (US)

(73) Assignee: Boston Scientific Scimed, Inc., Maple Grove, MN (US)

(21) Appl. No.: 18/966,502

(22) Filed: Dec. 3, 2024

Related U.S. Application Data
(60) Provisional application No. 63/605,824, filed on Dec. 4, 2023.

(51) Int. Cl. A61M 60/165; A61M 60/216; A61M 60/419; A61M 60/81; A61M 60/818

(52) U.S. Cl. CPC A61M 60/165; A61M 60/216; A61M 60/419; A61M 60/81; A61M 60/818; A61M 2205/0216; A61M 2205/04; A61M 2205/103

(57) ABSTRACT

A percutaneous circulatory support device including a percutaneous blood pump. The blood pump may include an impeller housing, an impeller disposed in the impeller housing, a bearing assembly, a motor housing, a motor disposed in the motor housing, and a flexible elongate shaft. The flexible elongate shaft may have a proximal portion coupled with the motor housing and a distal portion coupled with the bearing assembly. The flexible elongate shaft may have a first outer diameter between the motor housing and the bearing assembly and the impeller housing may have a second outer diameter that is greater than the first outer diameter.

### CROSS-REFERENCE TO RELATED APPLICATIONS

This application claims the benefit of U.S. Provisional Patent Application Ser. No. 63/605,824, filed Dec. 4, 2023, which is incorporated herein by reference.

### TECHNICAL FIELD

The present disclosure pertains to a circulatory support device. More particularly, the present disclosure pertains a percutaneous circulatory support device including a flexible connection between a motor housing and an impeller housing.

### BACKGROUND

Percutaneous mechanical circulatory support devices, such as blood pumps can provide transient support for hours or months of use in patients whose heart function or cardiac output is compromised. The percutaneous mechanical circulatory support devices may be sufficiently flexible to be navigated through the vasculature to a patient's heart. Such devices may be navigated through the aortic arch and placed across the aortic valve, for example. Various configurations of percutaneous mechanical circulatory support devices are known. However, there is an ongoing need to provide improved construction of percutaneous mechanical circulatory support devices.

### BRIEF SUMMARY

This disclosure provides design, material, manufacturing method, and use alternatives for medical devices, including percutaneous circulatory support devices and associated percutaneous blood pumps.

A first example is a blood pump comprising an impeller housing an impeller disposed in the impeller housing, a magnetic assembly in communication with the impeller, a motor housing, a motor disposed in the motor housing, wherein the motor is in communication with the magnetic assembly to drive the impeller, and a flexible elongate shaft coupled with the magnetic assembly and the motor housing, and wherein the flexible elongate shaft may have a first outer diameter that is less than a second outer diameter of the impeller housing.

Alternatively or additionally to any of the examples above, in another example, the motor housing may have a third outer diameter and the first outer diameter may be less than the third outer diameter.

Alternatively or additionally to any of the examples above, in another example, the flexible elongate shaft may have an elongate core member and an elongate casing extending along the elongate core member.

Alternatively or additionally to any of the examples above, in another example, the blood pump may further comprise a motor drive shaft, and wherein the elongate core member may have a proximal portion in communication with the motor drive shaft and a distal portion in communication with the magnetic assembly.

Alternatively or additionally to any of the examples above, in another example, the magnetic assembly may comprise a driven shaft and the elongate core member may have a distal portion in communication with the driven shaft.

Alternatively or additionally to any of the examples above, in another example, the magnetic assembly may be located in the impeller housing.

Alternatively or additionally to any of the examples above, in another example, the magnetic assembly may further comprise a driven magnet coupled with the impeller and a drive magnet fluidly isolated from the driven magnet and configured to drive the driven magnet.

Alternatively or additionally to any of the examples above, in another example, the driven magnet may be located in the impeller housing.

Alternatively or additionally to any of the examples above, in another example, the blood pump may further comprise a driven shaft having a proximal portion coupled with the flexible elongate shaft and a distal portion coupled with the drive magnet.

Alternatively or additionally to any of the examples above, in another example, the blood pump may further comprise a bearing assembly coupled with the impeller housing and the flexible elongate shaft.

In another example, a blood pump may comprise an impeller housing, an impeller disposed in the impeller housing, a bearing assembly coupled with the impeller housing, a motor housing, a motor disposed in the motor housing, and a flexible elongate shaft having a proximal portion coupled with the motor housing and a distal portion coupled with the bearing assembly.

Alternatively or additionally to any of the examples above, in another example, the flexible elongate shaft may have an elongate core member and an elongate casing extending along the elongate core member, the elongate casing may be coupled with the bearing assembly.

Alternatively or additionally to any of the examples above, in another example, the blood pump may further comprise a motor drive shaft, and wherein the elongate core member may have a proximal portion in communication with the motor drive shaft.

Alternatively or additionally to any of the examples above, in another example, the elongate core member may extend through the bearing assembly.

Alternatively or additionally to any of the examples above, in another example, the blood pump may further comprise a magnetic assembly coupled with the distal portion of the flexible elongate shaft, and wherein the magnetic assembly may be configured to drive the impeller in response to actuation of the motor.

Alternatively or additionally to any of the examples above, in another example, the magnetic assembly may comprise a driven magnet coupled with the impeller and a drive magnet fluidly isolated from the driven magnet and coupled with the distal portion of the flexible elongate shaft to drive the driven magnet in response to actuation of the motor.

Alternatively or additionally to any of the examples above, in another example, the blood pump may further comprise a driven shaft having a proximal portion extending through the bearing assembly and a distal portion coupled with the drive magnet.

Alternatively or additionally to any of the examples above, in another example, the flexible elongate shaft may have an elongate core member having a distal portion extending through the bearing assembly and coupled with the drive magnet.

Alternatively or additionally to any of the examples above, in another example, the elongate core member may comprise a flexible portion and a rigid portion, wherein the rigid portion extends through the bearing assembly and is coupled to the drive magnet.

In a further example, a blood pump may comprise an impeller housing, an impeller disposed in the impeller housing, a bearing assembly coupled with a proximal end of the impeller housing, a motor housing, a motor disposed in the motor housing, and a flexible elongate shaft having a proximal portion coupled with the motor housing and a distal portion coupled with the bearing assembly, and wherein the flexible elongate shaft may have a first outer diameter between the motor housing and the bearing assembly and the impeller housing may have a second outer diameter that is greater than the first outer diameter.

The above summary of some configurations is not intended to describe each disclosed configuration or every implementation of the present disclosure. The Figures, and Detailed Description, which follow, more particularly exemplify some of these configurations.

### BRIEF DESCRIPTION OF THE DRAWINGS

The disclosure may be more completely understood in consideration of the following detailed description in connection with the accompanying drawings, in which:

FIG. 1 is a schematic perspective view of an illustrative percutaneous circulatory support device including a percutaneous blood pump;

FIG. 2 is a schematic perspective view of the distal end region of the illustrative percutaneous circulatory support device of FIG. 1 including the percutaneous blood pump;

FIG. 3 is a schematic side view of a portion of the illustrative percutaneous blood pump of FIG. 1;

FIG. 4 is a schematic cross-sectional view of the portion of the illustrative percutaneous blood pump depicted in FIG. 3;

FIG. 5 is a schematic cross-sectional view of a portion of an illustrative percutaneous blood pump;

FIG. 6 is a schematic cross-sectional view of the portion of the illustrative percutaneous blood pump depicted in FIG. 5, with a bend in an elongate flexible member;

FIG. 7 is a schematic cross-sectional view of a portion of an illustrative percutaneous blood pump; and

FIG. 8 is a schematic cross-sectional view of a portion of an illustrative percutaneous blood pump.

While the disclosure is amenable to various modifications and alternative forms, specifics thereof have been shown by way of example in the drawings and will be described in detail. It should be understood, however, that the intention is not to limit the disclosure to the particular configurations described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an configuration”, “some configurations”, “other configurations”, etc., indicate that the configuration described may include one or more particular features, structures, and/or characteristics. However, such recitations do not necessarily mean that all configurations include the particular features, structures, and/or characteristics. Additionally, when particular features, structures, and/or characteristics are described in connection with one configuration, it should be understood that such features, structures, and/or characteristics may also be used connection with other configurations whether or not explicitly described unless clearly stated to the contrary.

The following detailed description should be read with reference to the drawings in which similar structures in different drawings are numbered the same. The drawings, which are not necessarily to scale, depict illustrative configurations and are not intended to limit the scope of the disclosure.

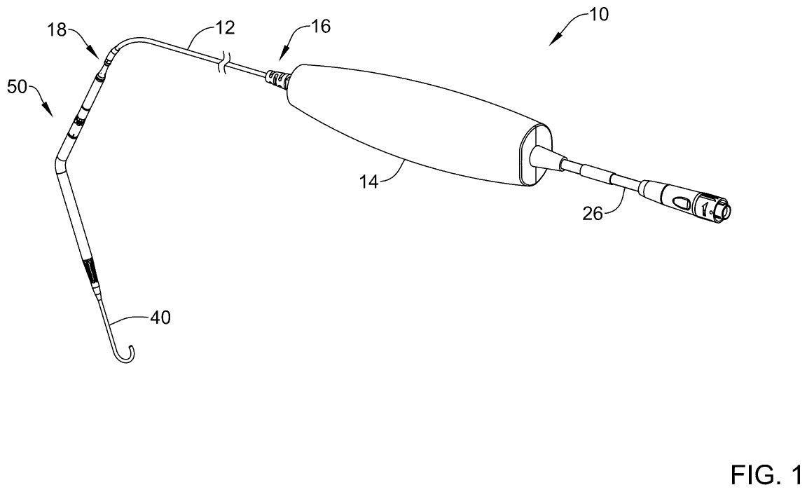

FIG. 1 illustrates a perspective view of an illustrative percutaneous circulatory support device 10 including a percutaneous blood pump 50 located at a distal end region thereof. The percutaneous circulatory support device 10 may be coupled to or include the blood pump 50, with an elongate shaft 12 of the percutaneous circulatory support device 10 extending proximally from the percutaneous blood pump 50 and a distal tip 40 extending distally from the blood pump 50. For instance, a proximal end 16 of the elongate shaft 12 may be coupled to a control module 14 and a distal end 18 of the elongate shaft 12 may be coupled to the percutaneous blood pump 50. An extension 26 may extend from the control module 14 to be connected to a controller (not shown) for controlling the blood pump 50, such as providing electrical power to the blood pump 50 and/or for sending and/or receiving signals, such as from one or more sensors during operation of the blood pump 50. In some cases, the extension 26 may transfer current to the blood pump 50 and/or electrical and/or optical signals from diagnostic sensors (such as, but not limited to, blood pressure, blood flow velocity, or the like).

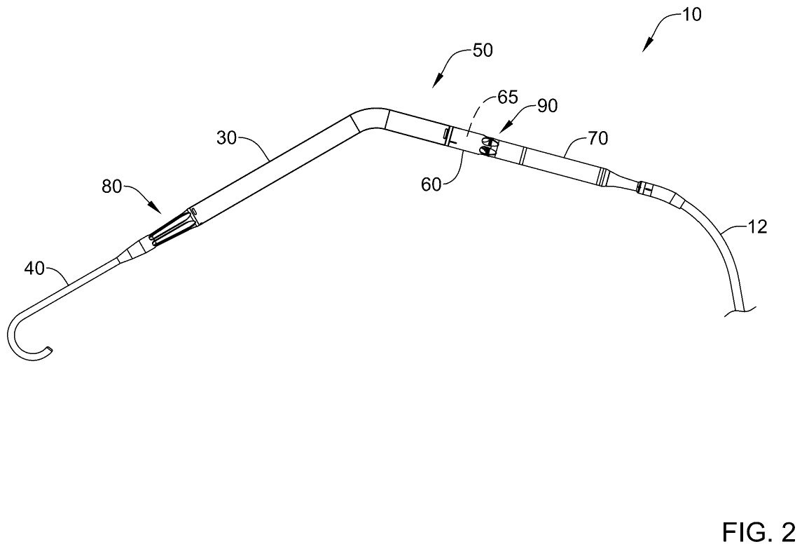

Additional features of the blood pump 50 are illustrated in FIG. 2. The blood pump 50 may generally include a flexible cannula 30, an impeller housing 60, and a motor housing 70. In some configurations, the flexible cannula 30, the impeller housing 60, and/or the motor housing 70 may be integrally or monolithically constructed. In other instances, the flexible cannula 30, the impeller housing 60, and/or the motor housing 70 may be separate components. The impeller housing 60 carries an impeller assembly 65 therein. The impeller assembly 65 may include an impeller secured to an impeller shaft, that rotates relative to the impeller housing 60 to drive blood through the blood pump 50. In some configurations, the impeller shaft and the impeller of the impeller assembly 65 may be integrally formed, whereas, in other configurations the impeller shaft and the impeller may be separate components.

Rotation of the impeller causes blood to flow from a blood inlet 80 of the blood pump 50, such as at a distal end of the flexible cannula 30, through the flexible cannula 30 and the impeller housing 60, and out of a blood outlet 90 proximal of the impeller, such as through a sidewall formed on the impeller housing 60. In some instances, the blood inlet 80 may include a plurality of blood inlet windows arranged around a circumference of the blood pump 50 (e.g., the flexible cannula 30). In some instances, the blood outlet 90 may include a plurality of blood outflow windows arranged around a circumference of the impeller housing 60. In other configurations, the inlet 80 and/or the outlet 90 may be formed on other portions of the blood pump 50.

With continued reference to FIG. 2, the motor housing 70 carries a motor configured to rotatably drive the impeller of the impeller assembly 65 relative to the impeller housing 60. Electrical power may be supplied to the motor through wiring extending through the elongate shaft 12, for example. In some instances, the motor may be physically connected to the impeller. For example, in some configurations the impeller may be mounted on a drive shaft or drive line of the motor. In other configurations, the impeller shaft may be directly or indirectly coupled to the drive shaft of the motor. In some instances, the drive assembly may include a magnetic coupling between the motor and the impeller. For example, a drive magnet may be mounted on the drive shaft or drive line of the motor. Rotation of the drive magnet causes rotation of a driven magnet, which is connected to the impeller assembly 65. More specifically, in configurations incorporating an impeller shaft, the impeller shaft and the impeller of the impeller assembly 65 are configured to rotate with the driven magnet. In other configurations, the motor may be coupled to the impeller assembly 65 via other components.

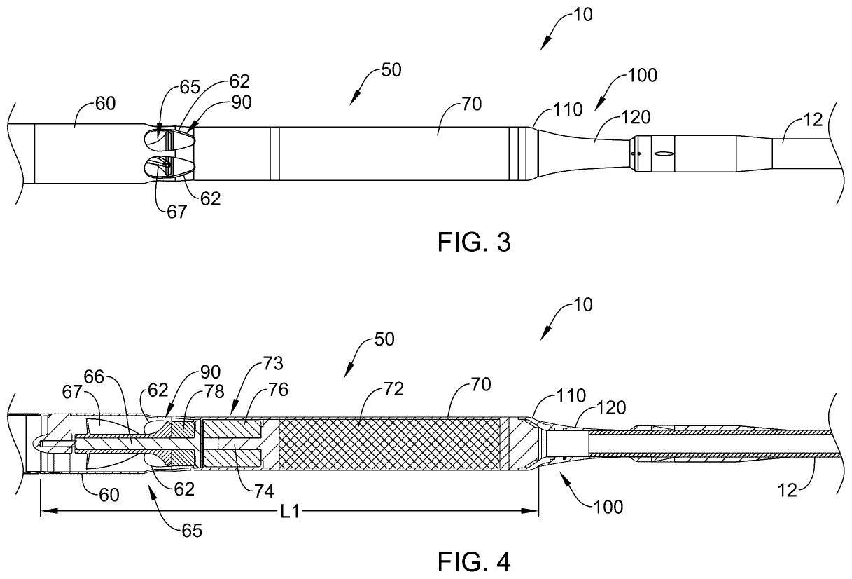

FIG. 3 is a side view of a portion of the percutaneous circulatory support device 10 illustrating a portion of the percutaneous blood pump 50 connected to the elongate shaft 12 at a junction 100. A proximal end of an impeller 67 of the impeller assembly 65 is also visible through the outflow openings 62 of the blood outlet 90.

The junction 100 may include an end cap 110 having a proximal end region surrounding the distal end region of the elongate shaft 12, and a fillet of material 120 surrounding the proximal end region of the end cap 110 and extending proximally therefrom. The fillet of material 120 may extend proximal of the end cap 110 and surround a portion of the elongate shaft 12 extending proximal of the end cap 110.

FIG. 4 is a cross-sectional view of the portion of the percutaneous circulatory support device 10 of FIG. 3, including the junction 100 between the percutaneous blood pump 50 and the elongate shaft 12 of the percutaneous circulatory support device 10. The junction 100 may be configured to mechanically connect the motor housing 70 (e.g., a metallic motor housing) of the blood pump 50, or other component of the blood pump 50, to the distal end region of the elongate shaft 12 (e.g., a polymeric tubular member).

As shown in FIG. 4, the impeller assembly 65 may include the impeller shaft 66 (e.g., a driven shaft) and an impeller 67 coupled thereto, where the impeller shaft 66 may be configured to rotate with the impeller 67. As shown, the impeller shaft 66 may be at least partially disposed within the impeller 67, but other suitable configurations are contemplated.

The impeller assembly 65 may further include a driven magnet 78 coupled to, and at least partially surrounding, the impeller shaft 66 and/or the impeller 67. The driven magnet 78 may be any type of magnetic rotor capable of being driven by a drive magnet 76. In this manner, as a magnetic field may be applied to the driven magnet 78 by the drive magnet 76 due to actuation of the motor 72, the driven magnet 78 may rotate, causing the impeller shaft 66 and impeller 67 to rotate.

As shown in FIG. 4, a magnetic assembly 73 may include the driven magnet 78 and the drive magnet 76 coupled to a drive shaft 74 (e.g., a motor drive shaft) configured to transfer torque from the motor 72 to the drive magnet 76. The motor housing 70 may be configured to hermetically seal (e.g., fluidly isolate) the drive magnet 76 and the motor within the motor housing 70, and thus fluidly isolate the drive magnet 76 from the driven magnet 78. Electrical wires, not shown, may extend through a lumen of the elongate shaft 12 to provide electrical power to the motor 72. In other instances, the drive shaft 74 may be directly coupled to the impeller 67 and/or impeller shaft 66, or other driven trains may be provided to transfer torque from the motor 72 to the impeller 67.

As depicted in FIG. 4, the length of the impeller housing 60 and the motor housing 70 may form a first rigid length of the blood pump 50. The first rigid length may be any suitable length. In some cases, the rigid length of the blood pump 50 in view of anatomy of a subject may determine where the blood pump 50 may be utilized in the subject, if at all, as the longer the rigid length, the more difficult it may be to position the blood pump 50 in the subject and maintain the blood pump 50 at a desired location.

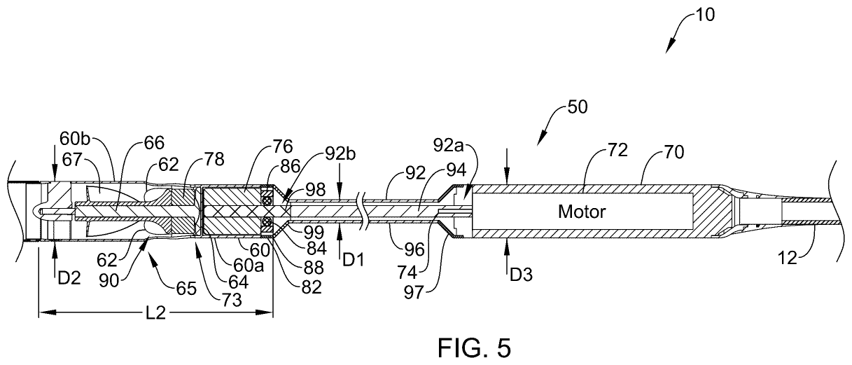

In some cases, the blood pump 50 may be configured with the impeller housing 60 and the motor housing 70 separated by a flexible elongate shaft, so as to reduce the length of a rigid portion of the blood pump 50 proximate a heart of the patient (e.g., reducing the rigid length). Reducing a length of the rigid portion of the blood pump 50 that is configured to be proximate the heart of the patient may facilitate inserting the blood pump 50 into the patient, tracking the blood pump 50 along vessels of the patient to a target site (e.g., a ventricle of the heart of the patient and/or other suitable target site), and/or operation of the blood pump 50. Further, the flexible elongate shaft between the impeller housing 60 and the motor housing 70 may include the drive shaft 74 and/or may include a flexible elongate drive core configured to couple the drive shaft 74 with the drive magnet 76 such that the motor 72 may drive the impeller 67 from a location spaced proximally from the impeller housing 60. FIGS. 5-7 depict portions of configurations of the percutaneous circulatory support device 10 with the blood pump 50, where the impeller housing 60 and the motor housing 70 are separated by a flexible elongate shaft 92.

FIG. 5 schematically depicts a cross-sectional view of a portion of the percutaneous circulatory support device 10, where the depicted components of the percutaneous circulatory support device 10 are axially aligned. As depicted in FIG. 5, the motor housing 70 may be spaced apart proximally from the magnetic assembly 73 and the impeller housing 60 by the flexible elongate shaft 92 and one or more bearing assemblies 82. As shown in FIG. 5, the flexible elongate shaft 92 may extend between the impeller housing 60 and the motor housing 70, with the one or more bearing assemblies 82 located distal of the flexible elongate shaft 92. In some examples, the flexible elongate shaft 92 may be coupled with the motor 72 and/or the motor housing 70 and the magnetic assembly 73. In operation, the impeller 67 may be driven in a manner similar to as discussed herein, where the motor 72 may be actuated and initiate rotation of the drive shaft 74, which may cause the drive magnet 76 to rotate in a manner that results in rotation of the driven magnet 78 and the impeller 67 in a desired direction and at a desired speed. The motor 72, the drive shaft 74, and the drive magnet 76 may be hermetically sealed (e.g., fluidly isolated) from the driven magnet 78 coupled with the impeller 67 (e.g., via the impeller shaft 66).

As depicted in FIG. 5, the impeller housing 60 may house the magnetic assembly 73 and the impeller 67. In some cases, the impeller housing 60 may include a divider 64 (e.g., wall and/or other suitable divider), where the divider 64 may hermetically seal the drive magnet 76 within a first portion 60a of the impeller housing 60, while the driven magnet 78 coupled to the impeller 67 via the impeller shaft 66 may be positioned within a second portion 60b of the impeller housing 60 through which blood may be outputted from the openings 62 thereof.

In some examples, the drive magnet 76 may be located within a housing that is formed separate from the impeller housing 60 and coupled to or coupled with the impeller housing 60. When separately formed, the housing in which the drive magnet 76 may be located may be coupled to a proximal end of the impeller housing 60 in any suitable manner including, but not limited to, using a welding technique, a soldering technique, and/or other suitable technique.

As discussed, the flexible elongate shaft 92 may be configured to directly or indirectly (e.g., via one or more components) couple the motor housing 70 with the impeller housing 60. Thus, the central axis of the impeller housing 60 (which also may be the rotational axis of the impeller 67) may be non-parallel to the central axis of the motor housing 70 (which also may be the rotational axis of the motor drive shaft 74). In some examples, the flexible elongate shaft 92 may comprise an elongate core member 94 (e.g., a flexible elongate core member 94) and an elongate casing 96 (e.g., a flexible elongate casing) extending along the elongate core member 94.

The flexible elongate shaft 92 may have any suitable length. Example suitable lengths of the flexible elongate shaft 92 include, but are not limited to, lengths less than about 7 centimeters (cm), lengths in a range of about 5 cm to about 12 cm, lengths in a range of about 7 cm to about 10 cm, lengths greater than 10 cm, lengths greater than 12 cm, and/or other suitable lengths.

In some examples, the length of the flexible elongate shaft 92 may be a suitable distance to allow the impeller housing 60 to be positioned distal of an aortic arch of a patient (e.g., in a left ventricle of the heart) and for the motor housing 70 to be positioned within the vasculature of the patient and proximal of the aortic arch. In some examples, the length of the flexible elongate shaft 92 may be a suitable distance to allow the impeller housing 60 to be positioned distal of the aortic arch of the patient and for the motor housing 70 to be positioned exterior of an access site into the patient. Other suitable length configurations for the flexible elongate shaft 92 are contemplated, such that a position of the motor housing 70 and the motor 72 relative to the impeller housing 60 may facilitate inserting the blood pump 50 into the patient, tracking the blood pump 50 along vessels of the patient to a target site (e.g., a ventricle of the heart of the patient and/or other suitable target site), and/or operation of the blood pump 50.

The elongate core member 94 may extend between the motor 72 and the drive magnet 76 to transfer rotational movement from the motor 72 to the drive magnet 76. The elongate core member 94 may be an elongate, flexible configuration of the drive shaft 74, but other suitable configurations of the elongate core member 94 are contemplated, as discussed herein and/or otherwise. In some example configurations, the elongate core member 94 may extend between and couple with the motor 72 and the drive magnet 76 to transfer movement from the motor 72 to the drive magnet 76 and drive the impeller 67 in response to actuation of the motor 72. The elongate core member 94 may be flexible such that the motor 72 and motor housing 70 may be positioned at a non-parallel angle to the impeller housing 60 such that the axis of rotation of the impeller 67 in the impeller housing 60 is non-parallel to the axis of rotation of the motor drive shaft 74 extending from the motor 72.

The elongate core member 94 may have a proximal portion that includes or is coupled to or with (e.g., is in communication with) the motor drive shaft 74 at a proximal end 92a of the flexible elongate shaft 92. The motor drive shaft 74 may be a rigid shaft not intended to flex or bend during use, whereas the elongate core member 94 may be a flexible shaft intended to flex or bend during use. In some examples, the elongate core member 94 may be welded, soldered, or otherwise secured to or with drive shaft 74 in a robust manner configured to withstand rotational forces required drive the impeller 67. As such, rotation of the motor drive shaft 74 rotates the elongate core member 94.

The elongate core member 94 may have a distal portion that includes or is coupled to or with (e.g., is in communication with) the magnetic assembly 73 (e.g., the drive magnet 76) at a distal end 92b of the flexible elongate shaft 92. The elongate core member 94 may be coupled to or with the magnetic assembly with a welded, soldered, or other secure connection that is configured to withstand rotational forces required to drive the impeller 67.

In some configurations, the elongate core member 94 may include and/or couple with a rigid shaft 98 (e.g., a driven shaft), where the rigid shaft 98 (e.g., a distal end of the rigid shaft 98) may be coupled to or with the drive magnet 76. Although the rigid shaft 98 may be part of the elongate core member 94 in some configurations (e.g., such that the elongate core member 94 includes a rigid portion, such as the rigid shaft 98, and a flexible portion proximal of the rigid shaft 98), the rigid shaft 98 may be part of the magnetic assembly 73 in some configurations and the elongate core member 94 may be coupled to or with (e.g., is in communication with) the rigid shaft 98 (e.g., coupled to or with a proximal end of the rigid shaft 98). When coupled to or with the rigid shaft 98, the elongate core member 94 may be welded, soldered, or otherwise secured to or with respect to a proximal portion of the rigid shaft 98 and a distal portion of the rigid shaft 98 may be welded, soldered, or otherwise secured to or with respect to the drive magnet 76. In examples, however, the rigid shaft 98 may be omitted. When included, the rigid shaft 98 may be configured to ensure rotation of the elongate core member 94 at the distal end 92b of the flexible elongate shaft 92 is axially aligned with the rotational movement of the drive magnet 76 and/or the impeller 67.

The elongate core member 94 may be formed from any suitable type of material. Example suitable types of materials include, but are not limited to, metals, polymers, stainless steel, nickel-titanium alloys (nitinol), polyetheretherketone (PEEK), and/or other suitable maters. In some configurations (e.g., when the elongate core member 94 is formed from a metal material, etc.), the elongate core member 94 may include a thin floating sleeve configured to facilitate containing lubricant along the elongate core member 94, where the thin sleeve may be formed from a polymer (polyurethane, polyethylene terephthalate (PET), etc.).

The elongate core member 94 may be formed from a single component or multiple components. In some examples, the core member 94 may be a single strand of stainless steel or nickel-titanium alloy. Alternatively, in some examples the core member 94 may include multiple elongated coils, wires, and/or windings of stainless steel, nick-titanium alloy, and/or other suitable materials. Other suitable configurations of the elongate core member 94 are contemplated.

The flexible elongate shaft 92 may include an elongate coating or casing 96 (e.g., a flexible elongate coating or casing) extending over and/or along at least a portion of a length of the elongate core member 94. The elongate casing 96 may extend over the elongate core member 94 to facilitate isolating the elongate core member 94 from fluids (e.g., blood, etc.) within the patient, preventing the rotating elongate core member 94 from engaging patient tissue, and/or maintaining lubrication about the elongate core member 94.

The elongate casing 96 may have any suitable outer diameter. In some examples, a first outer diameter D1 at the elongate casing 96 of the flexible elongate shaft 92 may be less than a second outer diameter D2 of the impeller housing 60, the first outer diameter D1 may be less than a third outer diameter D3 of the motor housing 70, or the first outer diameter D1 may be less than the second outer diameter D2 and the third outer diameter D3. In one example, the first outer diameter D1 may be nine (9) French (e.g., about three (3) millimeters (mm)). Other suitable configurations are contemplated.

The elongate casing 96 may be formed from any suitable material. Example suitable materials include, but are not limited to, metals, polymers, polyurethane, PET, and/or other suitable materials. In some examples, the elongate casing 96 may be a polymer tube or sleeve applied over the elongate core member 94. In some examples, the elongate casing 96 may be a polymer reflow of material along the elongate core member 94. Other suitable configurations of the elongate casing 96 are contemplated.

The elongate casing 96 may include and/or may be coupled to a proximal cap 97 and a distal cap 99. The proximal cap 97 and/or the distal cap 99 may extend longitudinally and/or radially outward from the elongate casing 96 and couple the elongate casing 96 with the motor housing 70, the impeller housing 60, and/or other suitable components of the blood pump 50. To mitigate catching on tissue and/or to otherwise facilitate inserting the blood pump 50 into vessels and/or traversing vessels of the patient, the proximal cap 97 may have an outer diameter that reduces radially inward as the proximal cap 97 extends distally and the distal cap 99 may have an outer diameter that expands radially outward as the distal cap 99 extends distally. In some cases, the proximal cap 97 and/or the distal cap 99 may facilitate coupling the elongate casing 96 with the motor housing 70 and the impeller housing 60 to hermetically seal the drive components (e.g., the motor 72, the drive shaft 74, the elongate core member 94, the drive magnet 76, and/or other suitable components) of the blood pump from fluid within the patient. Further, hermetically sealing the drive components may facilitate maintaining lubrication about the drive components during operation.

The proximal cap 97 and/or the distal cap 99 may be formed from the same material as or a different material than the material from which the elongate casing 96 is formed. In some examples, the proximal cap 97 and/or the distal cap 99 may be formed from a metal material, a polymer material, stainless steel, nickel-based superalloys, INCONEL, nickel-titanium alloys, nitinol, nickel cobalt alloys, MP35N, and/or other suitable materials. In one example, the proximal cap 97 and the distal cap 99 may be formed from stainless steel and coupled with a polymer material of the elongate casing 96 using a polymer reflow process, but other suitable configurations are contemplated. When the proximal cap 97 and the distal cap 99 are formed from one or more metallic materials, the proximal cap 97 may be coupled with the motor housing 70 with a solder or weld connection and the distal cap 99 may be coupled with the impeller housing 60 or other component with a solder or weld connection.

The one or more bearing assemblies 82 may be any suitable type of bearing assembly. For example, the bearing assembly 82 may be a radial bearing assembly, a thrust bearing assembly, a radial and thrust bearing assembly, an oil-embedded sleeve/flange bearing assembly, and/or one or more other suitable types of bearing assembly. The bearing assembly 82, as schematically depicted in FIG. 5, may include at least ball bearings 84 and a bearing cage 86, but additional or alternative components may be utilized. Further, the bearing assembly 82 may include a housing 88 (e.g., a bearing housing) for housing the ball bearings 84, the bearing cage 86, and/or other suitable components of the bearing assembly 82. Although not depicted in FIG. 5, the bearing assembly 82 may include an inner ring configured to extend between the elongate core member 94 and the ball bearings 84. When included, the inner ring may be separate from or part of the bearing cage 86.

The bearing assembly 82 may be formed from any suitable materials. For example, the ball bearings 84, the bearing cage 86, the housing 88, and/or other suitable components of the bearing assembly 82, if any, may be formed from a metal material, a ceramic material, a stainless-steel material, and/or other one or more other suitable materials.

The bearing assembly 82 may be coupled with the distal end 92b of the flexible elongate shaft 92 (e.g., via the elongate core member 94 and/or the distal cap 99) and the impeller housing 60. In some examples, the bearing assembly 82 may be part of or housed in or by the impeller housing 60 and/or the distal cap 99. When the distal cap 99 is formed from a metallic material and the impeller housing 60 is formed from a metallic material, the bearing assembly 82 may be coupled with the distal cap 99 and/or the impeller housing 60 with a weld connection, a solder connection, a pressed connection, a laser weld connection, and/or other suitable type of connection. When one or more of the distal cap 99 and the impeller housing 60 are formed from a polymer material, a reflow or other suitable polymer coupling technique may be used to couple the bearing assembly 82 with the impeller housing 60 and/or the distal cap 99. In one example configuration, the bearing assembly 82 may be a pressed part positioned in the impeller housing 60 and the distal cap 99, where the distal cap 99, the impeller housing 60, the housing 88 of the bearing assembly 82, and/or the bearing cage 86 may be welded together (e.g., at a seam between the proximal end of the impeller housing 60 and the distal cap 99). Other suitable connection techniques are contemplated.

The bearing assembly 82 may be coupled to a proximal end of the impeller housing 60, or otherwise located in the impeller housing 60, proximal of the drive magnet 76. The bearing assembly 82 may be located distal of the joint or connection between the flexible elongate core member 94 and the rigid shaft 98, with the rigid shaft 98 extending through the bearing assembly 82. In some cases, the bearing assembly 82 may include a proximal opening and a distal opening such that the rigid shaft 98 (e.g., a distal portion of the elongate core member 94 and/or other suitable portion) may extend through the bearing assembly 82 to the drive magnet 76 to which the elongate core member 94 is secured or coupled. As the rigid shaft 98 passes through the bearing assembly 82, the rigid shaft 98 may engage an inner surface of the bearing assembly 82 to facilitate rotating the rigid shaft 98 about an axis aligned with an axis of rotation extending through the drive magnet 76, the driven magnet 78, the impeller shaft 66 and/or the impeller 67.

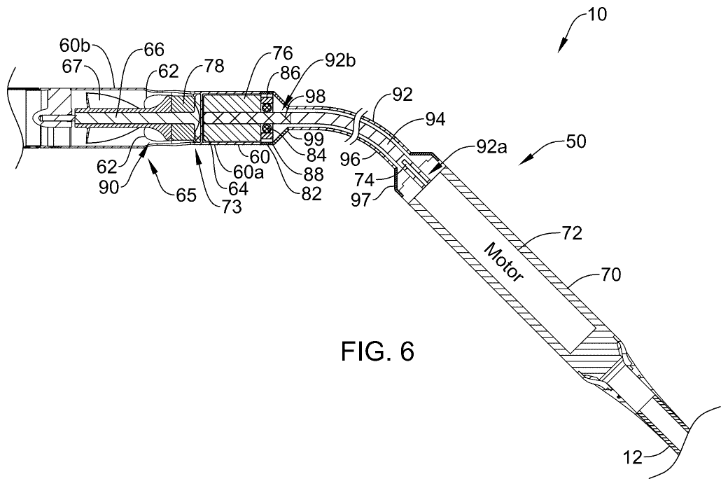

FIG. 6 schematically depicts the portion of the percutaneous circulatory support device 10 depicted in FIG. 5, where a bend is formed in the flexible elongate shaft 92. Although a single bend along the flexible elongate shaft 92 is depicted in FIG. 6, one or more bends may be formed in the flexible elongate shaft 92, as desired. When the flexible elongate shaft 92 is bent of flexed, the central axis of the impeller housing 60 (which also may be the rotational axis of the impeller 67) may be non-parallel to the central axis of the motor housing 70 (which also may be the rotational axis of the motor drive shaft 74).

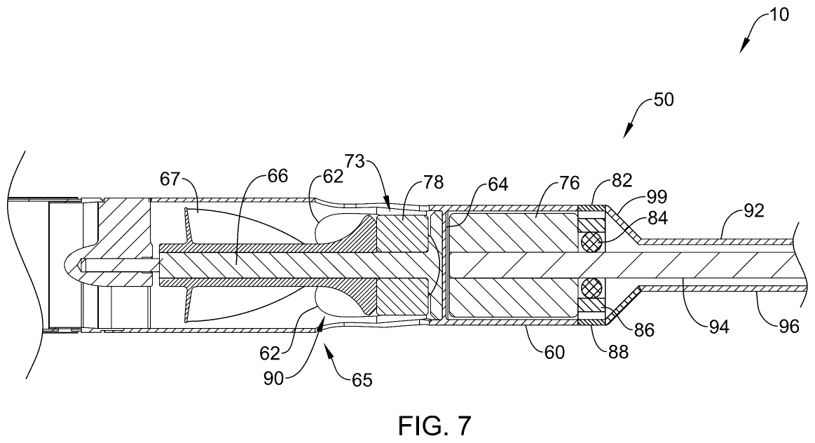

FIG. 7 schematically depicts a portion of the blood pump 50 of the percutaneous circulatory support device 10, where the elongate core member 94 may be formed from a single material and extend through the bearing assembly 82. In some examples, the single material of the elongate core member 94 may be a polymer material, a metallic material, and/or one or more other suitable materials. In one example, the single material of the elongate core member 94 may be a flexible polymer or metallic material and the bearing assembly 82 may facilitate ensuring the elongate core member 94 rotates about an axis aligned with an axis of the magnetic assembly 73, the impeller shaft 66, and the impeller 67 at a location proximate the drive magnet 76.

When the elongate core member 94 is formed from a polymer material, the elongate core member 94 may be formed from a first polymer and the elongate casing 96 may be formed from a second polymer that is the same as or different than the first polymer. In one example, the first polymer may be PEEK and/or other suitable material and the second polymer may be polyurethane, PET, and/or other suitable material, but other suitable configurations are contemplated. In some examples, a lubricant may be utilized between the elongate core member 94 (e.g., a metallic or polymer elongate core member) and the elongate casing 96 to minimize wear on the elongate core member 94 as it rotates in response to actuation of the motor 72.

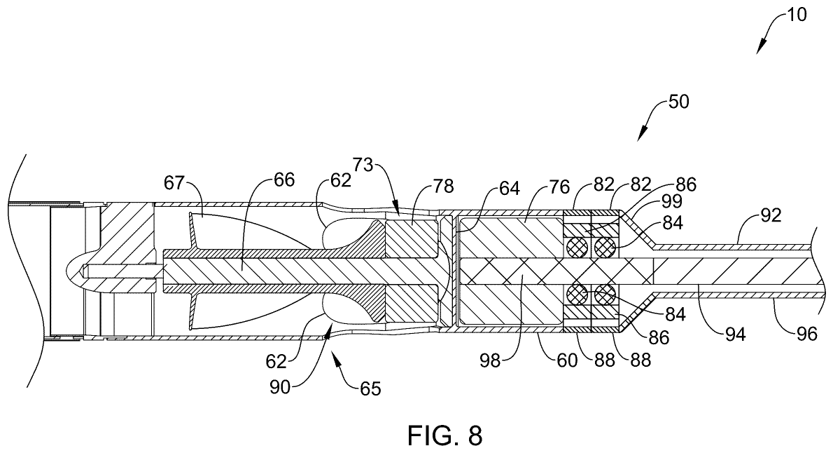

FIG. 8 schematically depicts a portion of the blood pump 50 of the percutaneous circulatory support device 10, where the blood pump 50 may include a plurality of bearing assemblies 82. As depicted in FIG. 8, the blood pump 50 may include two bearing assemblies 82, but other suitable number of bearing assemblies 82 may be utilized. Although the two bearing assemblies 82 in FIG. 8 are depicted as extending along the rigid shaft 98, it is contemplated that the flexible elongate core member 94 may extend through the bearing assemblies 82. In some examples, utilizing a plurality of bearing assemblies 82 and/or an axially elongate bearing assembly 82 may facilitate achieving a desired run-time durability as the plurality of bearing assemblies 82 and/or elongate bearing assembly 82 maintain stability of the rotating components of the blood pump 50 by centering the drive magnet 76 in the impeller housing 60 and mitigating contact between the drive magnet 76 and inner walls of the impeller housing 60 while the flexible elongate shaft 92 bends during a procedure and/or at other times.

The two or more bearing assemblies 82 may be positioned proximate one another and/or spaced from one another or at least spaced from one other bearing assembly 82 so as to maintain the drive magnet 76 in a central position within the impeller housing 60 or other suitable housing. As depicted in FIG. 8, the two bearing assemblies 82 may be positioned proximate one another and in axial alignment with one another. Alternatively, the two bearing assemblies may be spaced (e.g., axially spaced) from one another and in axial alignment with one another.

As discussed, the bearing assemblies 82 may include the ball bearings 84, the bearing cage 86, and/or the housing 88. In some examples, when the plurality of bearing assemblies 82 are proximate one another, each bearing assembly 82 may include the ball bearings 84, the bearing cage 86, and the housing 88. Alternatively, in some examples, when the plurality of bearing assemblies 82 are proximate one another, the bearing assembly 82 may share one or more components with one or more other bearing assemblies 82. In one example, a first bearing assembly 82 and a second bearing assembly 82 may have separate ball bearings 84, but share a single bearing cage 86 and/or housing 88. In one example, a first bearing assembly 82 and a second bearing assembly 82 may share one or more ball bearings 84 and have separate or shared bearing cages 86 and/or housings 88. In addition to or as an alternative to having multiple bearing assemblies 82, one or more bearing assemblies 82 may have elongated components that extend an axial length along the rigid shaft 98 or the flexible elongate core member 94 a distance greater than an axial length of a single, standard bearing assembly 82 (e.g., where a single standard bearing may be depicted in FIG. 5).

The plurality of bearing assemblies 82 may be coupled with each other, the distal end 92b of the flexible elongate shaft 92 (e.g., via the elongate core member 94 and/or the distal cap 99), and/or the impeller housing 60. In some examples, the plurality of bearing assemblies 82 may be part of or housed in or by the impeller housing 60 and/or the distal cap 99. When the housing 88 of the bearing assemblies 82 are formed from metallic material, the distal cap 99 is formed from a metallic material, and/or the impeller housing 60 is formed from a metallic material, the bearing assembly 82 may be coupled with each other, the distal cap 99, and/or the impeller housing 60 with a weld connection, a solder connection, a pressed connection, a laser weld connection, and/or other suitable type of connection. When one or more of the housing 88 of the bearing assemblies 82, the distal cap 99, and the impeller housing 60 are formed from a polymer material, a reflow or other suitable polymer coupling technique may be used to couple the bearing assembly 82 with each other, the impeller housing 60, and/or the distal cap 99. In one example configuration, the bearing assemblies 82 may be pressed parts positioned in the impeller housing 60 and the distal cap 99, where the distal cap 99, the impeller housing 60, the housing 88 of the bearing assemblies 82, and/or the bearing cages 86 may be welded together. Other suitable connection techniques are contemplated.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example configuration being used in other configurations. The scope of the disclosure is, of course, defined in the language in which the appended claims are expressed.

What is claimed is:

1. A blood pump comprising:
an impeller housing;
an impeller disposed in the impeller housing;
a magnetic assembly in communication with the impeller;
a motor housing;
a motor disposed in the motor housing, wherein the motor is in communication with the magnetic assembly to drive the impeller; and
a flexible elongate shaft coupled with the magnetic assembly and the motor housing, and
wherein the flexible elongate shaft has a first outer diameter that is less than a second outer diameter of the impeller housing.

2. The blood pump of claim 1, wherein the motor housing has a third outer diameter and the first outer diameter is less than the third outer diameter.

3. The blood pump of claim 1, wherein the flexible elongate shaft has an elongate core member and an elongate casing extending along the elongate core member.

4. The blood pump of claim 3, further comprising:
a motor drive shaft, and
wherein the elongate core member has a proximal portion in communication with the motor drive shaft and a distal portion in communication with the magnetic assembly.

5. The blood pump of claim 3, wherein the magnetic assembly comprises a driven shaft and the elongate core member has a distal portion in communication with the driven shaft.

6. The blood pump of claim 1, wherein the magnetic assembly is located in the impeller housing.

7. The blood pump of claim 1, wherein the magnetic assembly comprises:
a driven magnet coupled with the impeller; and
a drive magnet fluidly isolated from the driven magnet and configured to drive the driven magnet.

8. The blood pump of claim 7, wherein the driven magnet is located in the impeller housing.

9. The blood pump of claim 7, further comprising:
a driven shaft having a proximal portion coupled with the flexible elongate shaft and a distal portion coupled with the drive magnet.

10. The blood pump of claim 1, further comprising:
a bearing assembly coupled with the impeller housing and the flexible elongate shaft.

11. A blood pump comprising:
an impeller housing;
an impeller disposed in the impeller housing;
a bearing assembly coupled with the impeller housing;
a motor housing;
a motor disposed in the motor housing; and
a flexible elongate shaft having a proximal portion coupled with the motor housing and a distal portion coupled with the bearing assembly.

12. The blood pump of claim 11, wherein the flexible elongate shaft has an elongate core member and an elongate casing extending along the elongate core member, the elongate casing is coupled with the bearing assembly.

13. The blood pump of claim 12, further comprising:
a motor drive shaft, and
wherein the elongate core member has a proximal portion in communication with the motor drive shaft.

14. The blood pump of claim 12, wherein the elongate core member extends through the bearing assembly.

15. The blood pump of claim 11, further comprising:
a magnetic assembly coupled with the distal portion of the flexible elongate shaft, and
wherein the magnetic assembly is configured to drive the impeller in response to actuation of the motor.

16. The blood pump of claim 15, wherein the magnetic assembly comprises:
a driven magnet coupled with the impeller; and
a drive magnet fluidly isolated from the driven magnet and coupled with the distal portion of the flexible elongate shaft to drive the driven magnet in response to actuation of the motor.

17. The blood pump of claim 16, further comprising:
a driven shaft having a proximal portion extending through the bearing assembly and a distal portion coupled with the drive magnet.

18. The blood pump of claim 16, wherein the flexible elongate shaft has an elongate core member having a distal portion extending through the bearing assembly and coupled with the drive magnet.

19. The blood pump of claim 18, wherein the elongate core member comprises a flexible portion and a rigid portion, wherein the rigid portion extends through the bearing assembly and is coupled to the drive magnet.

20. A blood pump comprising:
an impeller housing;
an impeller disposed in the impeller housing;
a bearing assembly coupled with a proximal end of the impeller housing;
a motor housing;
a motor disposed in the motor housing; and
a flexible elongate shaft having a proximal portion coupled with the motor housing and a distal portion coupled with the bearing assembly, and
wherein the flexible elongate shaft has a first outer diameter between the motor housing and the bearing assembly and the impeller housing has a second outer diameter that is greater than the first outer diameter.
